(19) United States
(12) Patent Application Publication
(10) Pub. No.: US 2016/0317324 A1
(43) Pub. Date: Nov. 3, 2016

(54) SPINAL FUSION SURGERY INSTRUMENT FOR IMPLANTING AND INTERVERTEBRAL CAGE THEREOF

(71) Applicant: INTAI Technology Corporation, Taichung (TW)

(72) Inventors: Ming-Cheng CHO, Changhua City (TW); Dian-Ying LIN, Hemei Township (TW); Yung-Fang TSAI, Taichung City (TW); Shin-Chang CHUANG, Taichung City (TW); Hung-Yi CHEN, Wujie Township (TW)

(21) Appl. No.: 15/057,547

(22) Filed: Mar. 1, 2016

Related U.S. Application Data
(63) Continuation-in-part of application No. 14/828,266, filed on Aug. 17, 2015.

(30) Foreign Application Priority Data
Apr. 28, 2015 (TW) 104113539

(51) Int. Cl. A61F 2/46

(52) U.S. Cl. CPC A61F 2/4611; A61F 2002/4629

(57) ABSTRACT

A spinal fusion surgery instrument for implanting and an intervertebral cage thereof are provided. The spinal fusion surgery instrument includes a body, a gripper subassembly, a first control subassembly, and a second control subassembly. The first control subassembly is provided to generate a displacement between a first connecting member and a second connecting member of the gripper subassembly to change the angle of gripper of the intervertebral cage. The second control subassembly is provided to enable a first gripping member and a second gripping member of the gripper subassembly separating smoothly from the dowel pins of the intervertebral cage. By means of the operation model, the spinal fusion surgery instrument may be controlled easily, and the orientation of the intervertebral cage may be promoted in the process of the surgery.

### CROSS-REFERENCE TO RELATED APPLICATION

This application is a continuation-in-part of U.S. patent application Ser. No. 14/828,266, filed Aug. 17, 2015, which claims benefit of Taiwan Patent Application No. 104113539, filed Apr. 28, 2015, the disclosures of which are incorporated herein its entirety by reference.

### BACKGROUND OF THE INVENTION

1. Field of the Invention

The present invention generally relates to an implant instrument and an intervertebral cage thereof, in particular to the implant instrument and the intervertebral cage thereof which are feasible to be applied to the transforaminal lumbar interbody fusion (TLIF).

2. Description of the Related Art

Generally, the vertebrae disease is caused by long-term inappropriate pose, sport injury or comes with ages. When the intervertebral disk is damaged severely, two adjacent spine bones may approach with each other abnormally to suppress the surrounding nerves and the pain caused by the nerve compression may disable the patient from exercising.

Currently, the most common treatment is to implant an intervertebral cage into a location between two adjacent spine bones by means of an implant instrument, so as to increase or recover the distance between the two adjacent spine bones and avoid the nerves from being suppressed. In the case of the treatment, the clinical surgeon operates the discectomy and then implants the intervertebral cage into a location between two adjacent spine bones to recover the stability of the vertebrae. The TLIF generally has three manners of anterior, posterior and transforaminal lumbar, and the used intervertebral cage may vary with the manners. In practice, the transforaminal lumbar has advantages of less negative effect upon the surrounding tissues of the patient and shorter postoperative healing time, compared with the other surgery. However, as far as the surgeon is concerned, the transforaminal lumbar has a smaller operative field and a higher demand in terms of operating the surgical instruments. When the implant instrument and the intervertebral cage are implanted into the human body, the surgeon can only operate the surgical instruments according the instinct, touch and experience as it is difficult to do through visual contact. In addition, when the surgeon plans to separate the implant instrument from the intervertebral cage, even the intervertebral cage is placed in the accurate position, the intervertebral cage is easy to deviate from its accurate position because of the motion of the implant instrument, such that the repositioning is necessary and the surgery time has to be extended.

Moreover, conventional endoscope and surgery instrument for mini-invasive surgery are separate devices, so the operative field is easily shielded by these devices during the mini-invasive surgery, and it is hard for the surgeon to obtain more precise status (such as the relative location, the angle of deflection, or angle of rotation) of the distal end of the instruments through the smaller operative field. The surgeon can only operate the surgical instruments based on experience.

In addition, the surgery instrument with link structures is inevitably polluted by blood and tissue fluid after surgery, so it is necessary to wash the internal structural of the surgery instrument after surgery to avoid unsmooth operation in future. However, the conventional surgery instrument is not provided with a channel structure for easy to clean.

As a result, the inventor of the present invention provides a spinal fusion surgery instrument for implanting and an intervertebral cage thereof which aim to improve the shortcomings of the current technique, so as to promote the clinical or medical practicability.

### SUMMARY OF THE INVENTION

In view of the aforementioned problem, a primary objective of the present invention provides a spinal fusion surgery instrument for implanting and an intervertebral cage thereof which change an angle with respect to the location of the intervertebral cage by adjusting the relative position between each member of the gripper subassembly, so that the complicated operation of the implant instrument is simplified to reduce the arrangement time of the intervertebral cage.

In view of the aforementioned problem, a primary objective of the present invention provides a spinal fusion surgery instrument for implanting to reduce the expected factors such as elastic fatigue, delayed movement and so on of the elastic member or the flexible member by adjusting the relative movement between the stiffener members, such that the uncertainty in the operational process can be avoided. In addition, the first fastening slot and the second fastening slot of the jaw portion can separate from the gripping end of the intervertebral cage by enabling the stop pin resists against the guiding shapes of the first gripping member and the second gripping member, such that the technical problems such as vibration, displacement and so on can be resolved when the implant instrument releases the intervertebral cage.

In view of the aforementioned problem, a primary objective of the present invention provides an intervertebral cage of a spinal fusion surgery, wherein the guiding end of the body thereof is a streamline shape with a sharp angle, which is able to peel off or penetrate the surrounding tissues of the intervertebral disks effortlessly and effectively reduce the residual force formed between the intervertebral disks. The dowel pins disposed in parallel are able to be gripped by the gripper subassembly of the implant instrument, and also applied in the positioning by the C-arm during the process of the surgery, such that the implanted direction and angle of the intervertebral cage of the present invention can be adjusted immediately by the relative position of the dowel pins and the auxiliary dowel pin, so as to shorten the positioning time.

The present invention provides an implant instrument for gripping and implanting an intervertebral cage into a location between two adjacent spine bones. The intervertebral cage may include a gripping end and a guiding end. The implant instrument may include a body, a gripper subassembly, a first control subassembly, and a second control subassembly and an endoscope. The body may include a grasp rod and a fixed rod. The fixed rod has a first end and a second end, wherein the first end is disposed at the grasp rod, and the second end has a shape corresponding to the gripping end of the intervertebral cage. The fixed rod may be disposed with a first guiding slot and a second guiding slot which communicate with the second end. The fixed rod may have an outer thread near a side of the first end and may further include a straight limit slot near a side of the second end.

The gripper subassembly may include a first connecting member, a first gripping member, a second connecting member and a second gripping member. The first connecting member is connected to the first gripping member and disposed in the first guiding slot, and the second connecting member is connected to the second gripping member and disposed in the second guiding slot. The first connecting member has a first thread structure, the second connecting member has a second thread structure, the first gripping member has a first fastening slot, and the second gripping member has a second fastening slot, wherein the first fastening slot and the second fastening slot may form a jaw portion to grip the gripping end of the intervertebral cage. The first control subassembly may include a first rotating shaft and a second rotating shaft, the first rotating shaft may be connected to the second rotating shaft and sleeved outside the fixed rod, an internal thread of the first rotating shaft is screwed to the first thread structure, and an internal thread of the second rotating shaft is screwed to the second thread structure. The second control subassembly may include a sleeve and a gripping rotating shaft, and the sleeve is connected to the gripping rotating shaft and sheathed outside the fixed rod. The sleeve may further have a stop pin inserted into the limit slot, and an internal thread of the gripping rotating shaft is screwed to an outer thread of the fixed rod.

The endoscope is combined with the sleeve, and has an end facing the jaw part and provided with a camera.

When rotating the first control subassembly, the first connecting member and the second connecting member may respectively be moved to opposite directions along the first guiding slot and the second guiding slot to change a relative position between the jaw portion and the intervertebral cage so as to change an angle with respect to the location of the intervertebral cage. When the second control subassembly is rotated to move the sleeve toward the first end of the fixed rod, the stop pin resists against the first gripping member and the second gripping member for enabling the jaw portion open to release the intervertebral cage; otherwise, when the second control subassembly is rotated to move the sleeve toward the second end of the fixed rod, the jaw portion may be closed.

Preferably, the first guiding slot and/or the second guiding slot may further include a limit slot near the side of the first end of the fixed rod for limiting the operation of the gripper subassembly.

Preferably, the implant instrument of the present invention may further include a measuring unit for measuring a depth where the implant instrument inserts into vertebra. The measuring unit is sheathed outside the sleeve.

Preferably, the first thread structure, the second thread structure, the outer thread or a combination thereof may be a single thread or a multiple thread.

Preferably, the sleeve has an accommodating structure disposed on an outer surface thereof and defining a channel configured to accommodate the endoscope.

Preferably, the sleeve has a channel therein and configured to accommodate the endoscope, and the end of the endoscope is exposed out of an end of the channel close to the jaw part.

Preferably, the sleeve has a liquid inlet port disposed thereon and in communication with internal space thereof.

The present invention further provides an implant instrument for gripping and implanting an intervertebral cage into a location between two adjacent spine bones. The intervertebral cage may include a gripping end and a guiding end. The implant instrument may include a body, a gripper subassembly, a first control subassembly, and a second control subassembly. The body may include a grasp rod and a fixed rod. The fixed rod has a first end and a second end. The first end is disposed at the grasp rod, and the second end has a shape corresponding to the gripping end of the intervertebral cage. The fixed rod may be disposed with a first guiding slot and a second guiding slot which communicate with the second end. The fixed rod may have an outer thread near a side of the first end and may further include a straight limit slot near a side of the second end.

The gripper subassembly may include a first connecting member, a first gripping member, a second connecting member and a second gripping member. The first connecting member is connected to the first gripping member and disposed in the first guiding slot, and the second connecting member is connected to the second gripping member and disposed in the second guiding slot. The first connecting member has a first thread structure, the second connecting member has a second thread structure, the first gripping member has a first fastening slot, and the second gripping member has a second fastening slot, wherein the first fastening slot and the second fastening slot may form a jaw portion to grip the gripping end of the intervertebral cage. The first control subassembly may include a first rotating shaft and a second rotating shaft, the first rotating shaft may be connected to the second rotating shaft and sleeved outside the fixed rod, an internal thread of the first rotating shaft is screwed to the first thread structure, and an internal thread of the second rotating shaft is screwed to the second thread structure. The second control subassembly may include a sleeve and a gripping rotating shaft, and the sleeve is connected to the gripping rotating shaft and sheathed outside the fixed rod. An internal thread of the gripping rotating shaft is screwed to the outer thread of the fixed rod. The sleeve has a liquid inlet port disposed thereon and in communication with internal space thereof.

When rotating the first control subassembly, the first connecting member and the second connecting member may respectively be moved to opposite directions along the first guiding slot and the second guiding slot to change a relative position between the jaw portion and the intervertebral cage so as to change an angle with respect to the location of the intervertebral cage. When the second control subassembly is rotated to move the sleeve toward the first end of the fixed rod, the stop pin resists against the first gripping member and the second gripping member for enabling the jaw portion open to release the intervertebral cage; otherwise, when the second control subassembly is rotated to move the sleeve toward the second end of the fixed rod, the jaw portion may be closed.

Preferably, the implant instrument further includes an endoscope combined with the sleeve, and having an end provided with a camera and facing the jaw part.

Preferably, the sleeve has an accommodating structure disposed on an outer surface thereof and defining a channel configured to accommodate the endoscope.

Preferably, the sleeve has a channel therein and configured to accommodate the endoscope, and the end of the endoscope is exposed out of an end of the channel close to the jaw part.

### BRIEF DESCRIPTION OF THE DRAWINGS

The detailed structure, operating principle and effects of the present invention will now be described in more details hereinafter with reference to the accompanying drawings that show various embodiments of the disclosure as follows.

FIG. 1 is a schematic diagram of the spinal fusion surgery instrument for implanting and the intervertebral cage thereof of a first embodiment of the present invention.

FIG. 2 is an explosion diagram for showing the spinal fusion surgery instrument for implanting of the present invention.

FIG. 3 is a schematic diagram of the intervertebral cage of the present invention.

FIG. 4 is a schematic diagram for showing the placement operations of the intervertebral cage of the present invention.

FIG. 5 is a schematic diagram for showing the relief operations of the intervertebral cage of the present invention.

FIG. 6 is a schematic diagram for showing the Transforaminal Lumbar Interbody Fusion by means of the spinal fusion surgery instrument for implanting and the intervertebral cage thereof of the present invention.

FIG. 7 is a schematic diagram of the spinal fusion surgery instrument for implanting of a second embodiment of the present invention.

FIG. 8 is a schematic diagram of the spinal fusion surgery instrument for implanting of a third embodiment of the present invention.

### DETAILED DESCRIPTION OF THE PREFERRED EMBODIMENTS

Hereinafter, embodiments of the present invention will be described in detail with reference to the accompanying drawings so that those skilled in the art to which the present invention pertains can realize the present invention. As those skilled in the art would realize, the described embodiments may be modified in various different ways, all without departing from the spirit or scope of the present invention.

The exemplary embodiments of the present invention will be understood more fully from the detailed description given below and from the accompanying drawings of various embodiments of the disclosure, which, however, should not be taken to limit the disclosure to the specific embodiments, but are for explanation and understanding only.

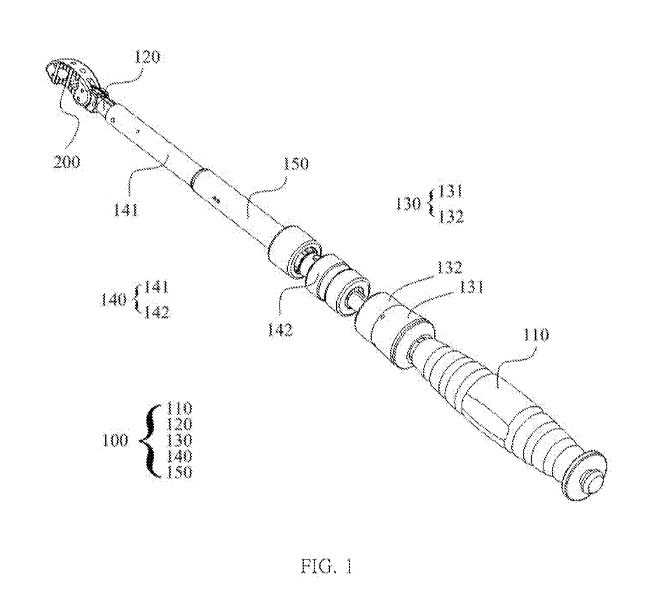

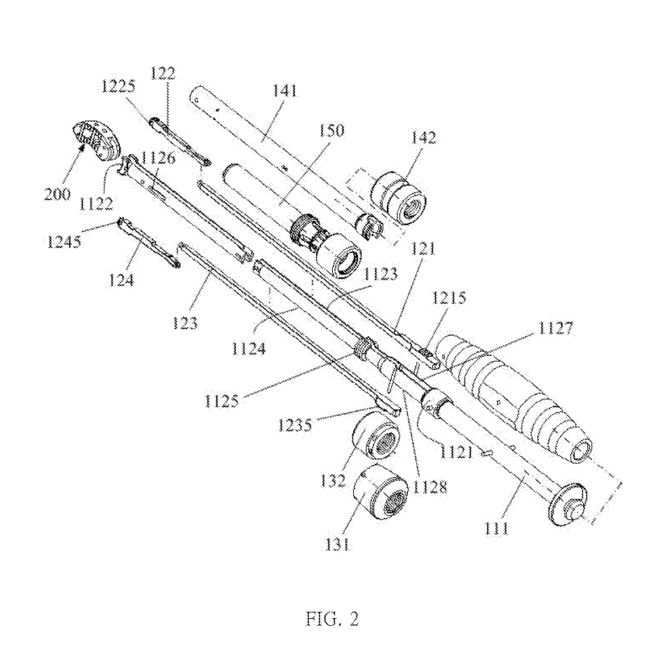

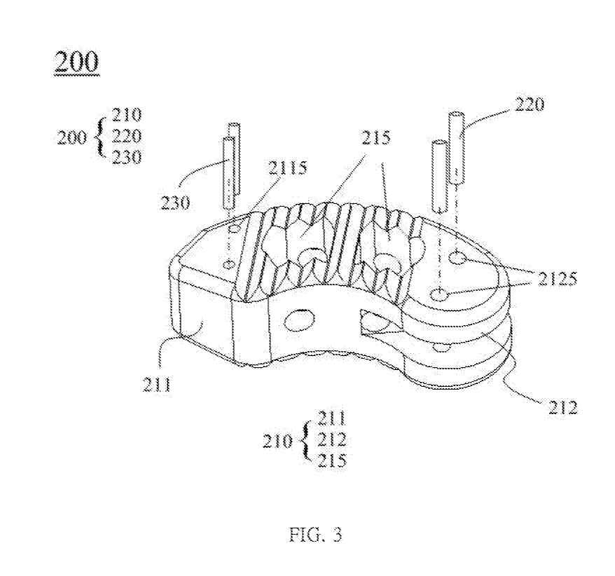

Please refer to FIG. 1, FIG. 2, and FIG. 3 which respectively show a spinal fusion surgery instrument for implanting and an intervertebral cage thereof of a first embodiment of the present invention. As shown in the FIGS., an implant instrument 100, which may be a spinal fusion surgery instrument for implanting, of the present invention is applied to grip and implant an intervertebral cage 200 to a location between two adjacent spine bones. The intervertebral cage 200 has a guiding end 211 and a gripping end 212. The implant instrument 100 includes a body 110, a gripper subassembly 120, a first control subassembly 130, a second control subassembly 140 and a measuring unit 150. The measuring unit 150 is sheathed outside the sleeve 141 of the second control subassembly 140. The body 110 includes a grasp rod 111 and a fixed rod 112. The gripper subassembly 120 includes a first connecting member 121, a first gripping member 122, a second connecting member 123 and a second gripping member 124. The first control subassembly 130 includes a first rotating shaft 131 and a second rotating shaft 132. The second control subassembly 140 includes the sleeve 141 and a gripping rotating shaft 142. The fixed rod 112 has a first end 1121 and a second end 1122. The first end 1121 may be assembled on the grasp rod 111 by means of screwing, sleeving, and so on. The second end 1122 has a shape corresponding to the gripping end 212. The fixed rod 112 has an outer thread 1125 disposed on a portion thereof near the first end 1121.

More precisely, a length of the fixed rod 112 is designed upon the necessary operative field and the operating space based on the injured area. When the required length of the fixed rod 112 is short, the fixed rod 112 may be formed integrally. The fixed rod 112 with the shorter length has a better precise operation, but the operative field may be easily shielded by the surgical instruments. For the sake of achieving a better operative field, the fixed rod 112 is preferably designed with a longer length, as such with an assembly-type fixed rod 112 may be operated. The diagrams of the present invention apply an assembly-type fixed rod 112 as an exemplary embodiment. In order to obtain the better operative field and facilitate to process and assemble the fixed rod 112, the dual structures of the fixed rod 112 may be assembled from the two sides of the sleeve 141 respectively, and then a pin is used to fix the dual structure of the fixed rod 112 as integrity. In practice, the first connecting member 121 and the second connecting member 123 may be integral or assembly-type upon the actual requirements.

The fixed rod 112 is disposed with a first guiding slot 1123 and a second guiding slot 1244 in communication with the second end 1122. In practice, the first guiding slot 1123 and the second guiding slot 1124 are respectively disposed at an upper side and a lower side of the fixed rod 112 in parallel, so that the fixed rod 112 is shaped as a H-shaped elongate shaft as a whole. Moreover, the first guiding slot 1123 and the second guiding slot 1124 respectively include limit slots 1127, 1128 formed near the first end 1121. The first connecting member 121 is connected to the first gripping member 122, disposed in the first guiding slot 1123, and linearly movable along the first guiding slot 1123. Similarly, the second connecting member 123 is connected to the second gripping member 124, disposed in the second guiding slot 1124, and linearly movable along the second guiding slot 1124. In practice, the lengths of the limit slots 1127, 1128 may be applied to respectively limit the range of motion of the first connecting member 121 and the second connecting member 123.

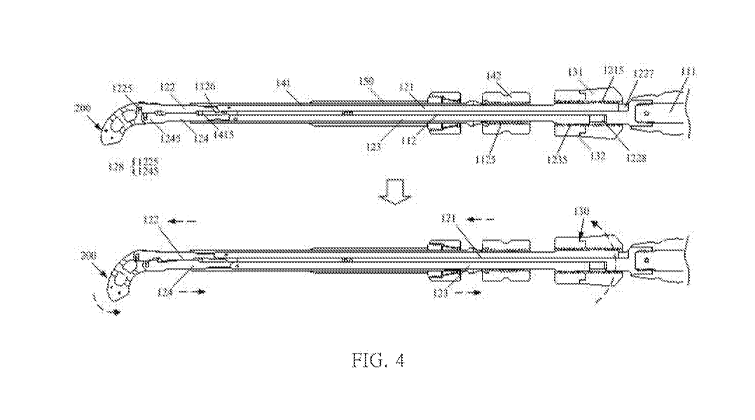

Please refer to FIG. 1 and FIG. 4 together. As shown in the FIGS., the first rotating shaft 131 of the first control subassembly 130 is connected to the second rotating shaft 132 to rotate simultaneously therewith. The first control subassembly 130 is sleeved outside the fixed rod 112 for being used by a surgeon. An internal thread of the first rotating shaft 131 is screwed to the first thread structure 1215 disposed at a distal end of the first connecting member 121, and an internal thread of the second rotating shaft 132 is also screwed to a second thread structure 1235 of the second connecting member 123. When the surgeon rotates the first control subassembly 130, the first connecting member 121 and the second connecting member 123 respectively are moved in opposite directions along the first guiding slot 1123 and the second guiding slot 1124 of the fixed rod 112, towards two sides of the fixed rod 112. In practice, the first thread structure 1215, the second thread structure 1235, the outer thread 1125 of the fixed rod 112 or a combination thereof may be a single thread characterized of accurate motion or a multiple thread which is applied to increase the operating space and shorten the rotation time.

To be precise, the first gripping member 122 of the gripper subassembly 120 has a first fastening slot 1225, and the second gripping member 124 of the gripper subassembly 120 has a second fastening slot 1245. The first fastening slot 1225 and the second fastening slot 1245 are applied to form a jaw part 128 to grip the gripping end 212 of the intervertebral cage 200. In practice, the first fastening slot 1225 and the second fastening slot 1245 are disposed in parallel to grip a dowel pin 220 of the intervertebral cage 200. For example, while the first control subassembly 130 is being rotated, the first connecting member 121 is moved towards the second end 1122 along the first guiding slot 1123 and the second connecting member 123 is moved towards the first end 1121 along the second guiding slot 1124. The opposite motion between the first connecting member 121 and the second connecting member 123 changes an angle with respect to the location of the intervertebral cage 200.

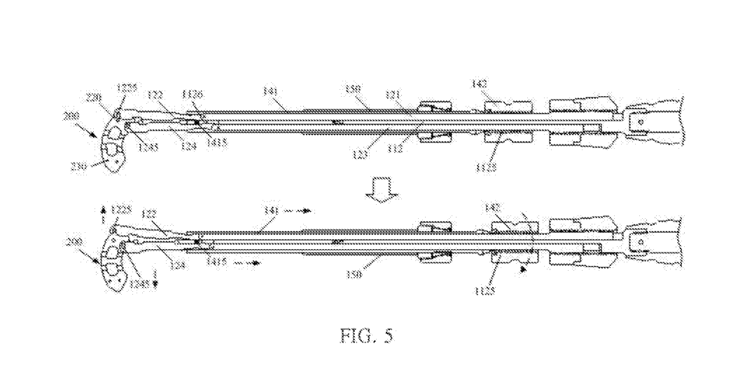

Please refer to FIG. 1 and FIG. 5 together. The sleeve 141 of the second control subassembly 140 is connected to the gripping rotating shaft 142 and sheathed outside the fixed rod 112. An internal thread of the gripping rotating shaft 142 is screwed to an outer thread 1125 of the fixed rod 112. When the surgeon rotates the second control subassembly 140, the sleeve 141 is moved linearly toward the first end 1121 or the second end 1122 along the fixed rod 112 upon the rotation direction, so as to close or open the jaw part 128. Furthermore, the fixed rod 112 further includes a straight limit slot 1126 near a side of the second end 1122 thereof, and the sleeve 141 further includes a stop pin 1415 which is inserted into the straight limit slot 1126. While the second control subassembly 140 is being rotated to move the sleeve 141 towards the first end 1121 of the fixed rod 112, the stop pin 1415 resists against the guiding shapes of the first gripping member 122 and the second gripping member 124, such that the first fastening slot 1225 and the second fastening slot 1245 of the jaw part 128 can be separated from the dowel pin 220 of the gripping end 212 smoothly. As a result, technical problems that the intervertebral cage is vibrated and displaced easily while being released by using the conventional instrument, can be resolved. In practice, the ends of the first gripping member 122 and the second gripping member 124 may be designed as the corresponding guiding shapes upon the actual requirements.

Please refer to FIG. 1 and FIG. 3 together. The present invention further provides an intervertebral cage 200 which includes a body 210, two dowel pins 220, and an auxiliary dowel pin 230. The body 210 has a structure comprising a plurality of penetration cavities 215 which are mutual interlaced, and the body 210 has upper and lower surfaces with uneven surfaces to increase the contact area between the two spine bones and the intervertebral cage 200, such that the displacement or separation of the intervertebral cage 200 caused by external force may be avoided after the intervertebral cage 200 is implanted. In practice, the penetration holes formed by the penetration cavities 215 enable the patient's tissue (such as bone cells, nerves, blood vessels and so on) to penetrate or adhere therebetween, so as to promote the efficiency of the healing. However, if a removal ratio of the volume of the body 210 by the penetration cavities 215 is too high, the entire strength of the intervertebral cage 200 may be affected and the structure thereof may even be damaged. When the removal ratio is too small, the degree of the self-healing of the patient's cells may be affected. As a result, the plurality of penetration cavities 215 remove the volume of the body 210 by 30-70%, and preferably, by 35-65%.

The body 210 may be a bent formation such as peas or a meniscus, or a bullet shape. The body 210 includes a guiding end 211 and a gripping end 212. The guiding end 211 is a streamline shape with a sharp angle, which is able to peel off or penetrate the surrounding tissues of the intervertebral disks effortlessly and effectively reduce the residual force formed between the intervertebral disks. In practice, the body 210 is shaped with circumferential chamfers without a sharp shape, so as to avoid damaging surrounding tissues such as nerves, blood vessels and so on accidentally in the process of the surgery. The gripping end 212 of the body 210 is disposed with two dowel holes 2125 in parallel and the guiding end 211 is disposed with at least one auxiliary dowel hole 2115. The two dowel pins 220 and the auxiliary dowel pin 230 are penetrated in the corresponding holes arranged on the body 210. The two dowel pins 220 disposed in parallel is not only able to be gripped by the gripper subassembly 120 of the implant instrument 100, but also applied in the positioning by the C-arm during the process of the surgery. The intervertebral cage 200 of the present invention applies a relative position between the two dowel pins 220 and the auxiliary dowel pin 230 to promptly correct the guiding direction and angle in which the intervertebral cage 200 are implanted, so as to shorten the positioning time. In practice, usage of various numbers and diameters of the dowel pins 220 and the auxiliary dowel pin 230 may facilitate the surgeon to see dynamic relative positions of the implant instrument 100 and the intervertebral cage 200 more clearly in the process of the surgery, so as to enhance the positioning accuracy in the process of the surgery.

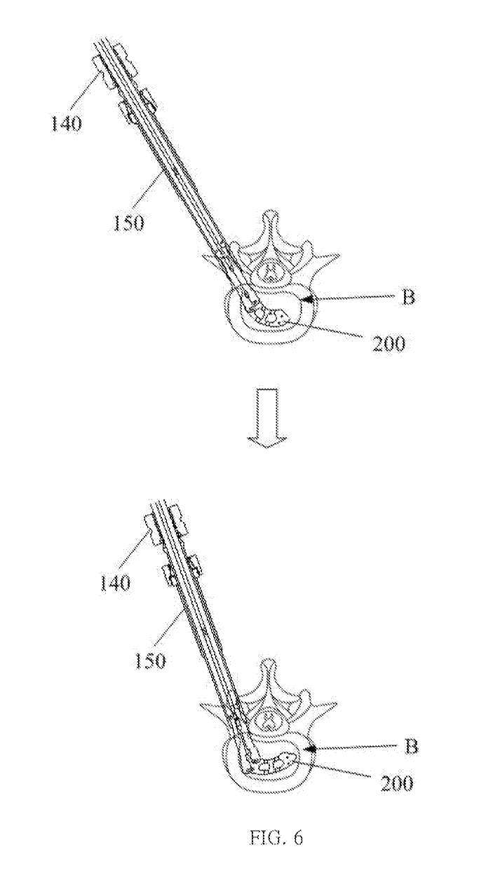

Please refer to FIG. 4, FIG. 5, and FIG. 6 together. While holding the implant instrument 100 to grip and implant the intervertebral cage 200 to the mounting position of the intervertebral foramen B, the surgeon implants the implant instrument 100 into the human body slowly until the intervertebral cage 200 contacts the surface of the spine bone, the surgeon releases and moves the measuring unit 150 to the scale zero and then re-blocks and re-fixes on the sleeve 141 to complete the step of positioning calibration. While the surgeon keeps pushing the intervertebral cage 200, the scales of the measuring unit 150 displays a depth where the implant instrument 100 and the intervertebral cage 200 are inserted into vertebra.

After confirming the intervertebral cage 200 to be located in the mounting position through the display device (not shown) and the measuring unit 150, the surgeon holds the measuring unit 150 in one hand and rotates the first control subassembly 130 by the other hand to change the gripping positions between the implant instrument 100 and the intervertebral cage 200, so as to rotate the intervertebral cage 200 for fitting the intervertebral cage 200 into an optimal mounting position. Moreover, when the intervertebral cage 200 is located in the mounting position, the surgeon can operate the second control subassembly 140 to separate the intervertebral cage 200 from the implant instrument 100 smoothly.

The implant instrument of the present invention is able to steplessly adjust an angle with respect to the location of the intervertebral cage by adjusting the relative positions between each member of the gripper subassembly, such that the surgeon can operate instinctively in the process of the surgery. Moreover, the implant instrument of the present invention can be operative to release the intervertebral cage 200 smoothly to reduce the positioning error. The intervertebral cage of the present invention further applies the relative positions between the two dowel pins and the auxiliary dowel pin to promptly correct the direction and angle in which the intervertebral cage is implanted, so as to shorten the positioning time.

Furthermore, the actuating members of the conventional implant instrument are usually elastic members or flexible members which easily cause uncertainty in the operational process due to unexpected factors such as elastic fatigue, delayed movement and so on, but the implant instrument of the present invention is able to reduce the unexpected factors by adjusting the relative movement between the rigid members, such that the uncertainty in the operational process can be avoided.

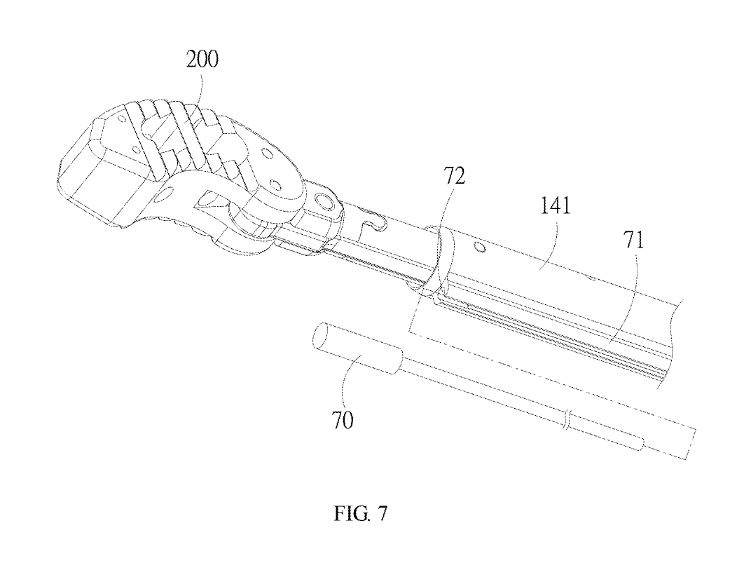

Please refer to FIG. 7 which shows a schematic view of an implant instrument of a second embodiment of the present disclosure. The difference between the second embodiment and above-described embodiment is that the implant instrument of the second embodiment further includes an endoscope 70. While the surgeon operates the implant instrument 100 to grip and implant the intervertebral cage 200 to the location between two adjacent spine bones, the endoscope 70 is configured to capture the image of the intervertebral cage 200, and transmit the image to an external display, so that the surgeon can directly watch the image shown on the external display during the surgery to clearly check the rotation status of the intervertebral cage 200 and a condition of the implantation area.

In the second embodiment, the sleeve 141 has an accommodating structure 71 disposed on an outer surface thereof and defining a channel inside to accommodate the endoscope 70. An end of the endoscope 70, which is provided with a camera, is exposed out of an end of the channel 72 close to the jaw part 128, and other end of the endoscope 70 is electrically connected to the external display. By means of combining the endoscope 70 with the implant instrument 100, the endoscope 70, the implant instrument 100 and the intervertebral cage 200 can be inserted to the implantation area at the same time, and the endoscope 70 can capture the image of the intervertebral cage 200 and transmit the captured image to the external display, to facilitate the surgeon to directly watch the image shown on the external display during the surgery to clearly check the rotation status of the intervertebral cage 200 and the condition of the implantation area.

The operation and structure of the endoscope is well known by those have skill in the art, so the detailed description is omitted. It should be noted that the structure of above-described endoscope 70 is just an example for illustration, but the present disclosure is not limited thereto. Any kinds of the endoscopes, which can be combined with the implant instrument 100 to capture the image of the intervertebral cage 200, should be in the scope of the appended claims.

In addition, the way of combining the endoscope 70 and the implant instrument 100 is not limited to above-described embodiment. For example, the sleeve 141 can has a channel formed inside to accommodate the endoscope, and two ends of the channel both are open ends, and an end of the endoscope, which is provided with the camera, can be exposed out of an end of the channel close to the jaw part 128, and other end of the endoscope can be electrically connected to the external display. Moreover, the way of combining the endoscope 70 on the outer surface of the sleeve 141 is not limited to the structure shown in FIG. 7. For example, the endoscope 70 can be adhered, fastened or locked on the outer surface of the sleeve 141.

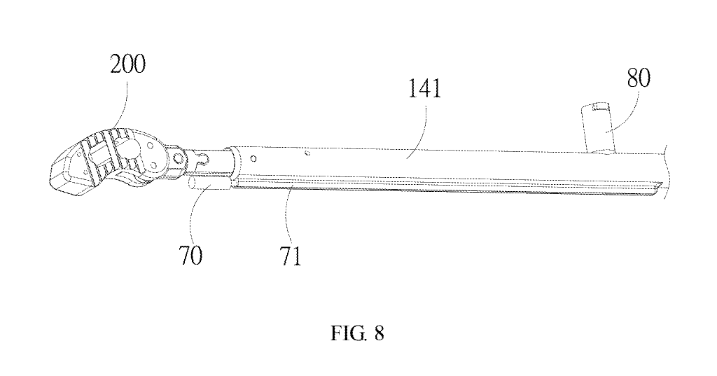

Please refer to FIG. 8 which shows a schematic view of an implant instrument of a third embodiment of the present disclosure. The difference between the third embodiment and above-described embodiments is that the sleeve 141 of the third embodiment further has a liquid inlet port 80 in communication with the inner space of the sleeve 141. Through the liquid inlet port 80, cleaning fluid can be inputted into the inner space of the sleeve 141 to wash the internal structure of the surgery instrument, so as to facilitate the cleaning and maintenance of the surgery instrument before or after the surgery.

While the means of specific embodiments in present invention has been described by reference drawings, numerous modifications and variations could be made thereto by those skilled in the art without departing from the scope and spirit of the disclosure set forth in the claims. The modifications and variations should in a range limited by the specification of the present invention.

What is claimed is:

1. An implant instrument operative to grip and implant an intervertebral cage into a location between two adjacent spine bones, the intervertebral cage comprising a gripping end and a guiding end, and the implant instrument comprising:
a body comprising a grasp rod and a fixed rod, the fixed rod having a first end and a second end, the first end configured to assemble with the grasp rod, the fixed rod having a first guiding slot and a second guiding slot in communication with the second end, and the fixed rod having an outer thread near a side of the first end;
a gripper subassembly comprising a first connecting member, a first gripping member, a second connecting member and a second gripping member, the first connecting member connected to the first gripping member and disposed in the first guiding slot, the second connecting member connected to the second gripping member and disposed in the second guiding slot, the first connecting member having a first thread structure and the second connecting member having a second thread structure, the first gripping member having a first fastening slot and the second gripping member having a second fastening slot, wherein the first fastening slot and the second fastening slot are configured to form a jaw portion to grip the gripping end of the intervertebral cage;
a first control subassembly comprising a first rotating shaft and a second rotating shaft, the first rotating shaft connected to the second rotating shaft and sleeved outside the fixed rod, the first rotating shaft having an internal thread configured to screw with the first thread structure and the second rotating shaft having an internal thread configured to screw with the second thread structure; and
a second control subassembly comprising a sleeve and a gripping rotating shaft, the sleeve connected to the gripping rotating shaft and sheathed outside the fixed rod, the gripping rotating shaft having an internal thread configured to screw with the outer thread of the fixed rod;
an endoscope combined with the sleeve and having an end facing the jaw part and provided with a camera;
wherein while the first control subassembly is rotated, the first connecting member and the second connecting member respectively are moved to opposite directions along the first guiding slot and the second guiding slot to change a relative position between the jaw portion and the intervertebral cage so as to change an angle with respect to the location of the intervertebral cage.

2. The implant instrument of claim 1, wherein when rotating the second control subassembly, the sleeve is moved linearly along the fixed rod to open or close the jaw portion.

3. The implant instrument of claim 1, wherein the fixed rod further comprises a straight limit slot near a side of the second end, and the sleeve further comprises a stop pin, wherein the stop pin is inserted into the straight limit slot, when rotating the second control subassembly to move the sleeve toward the first end of the fixed rod, and the stop pin resists against the first gripping member and the second gripping member, such that the jaw portion opens to release the intervertebral cage.

4. The implant instrument of claim 1, wherein the first guiding slot and/or the second guiding slot further comprise a limit slot near the side of the first end of the fixed rod for limiting operation of the gripper subassembly.

5. The implant instrument of claim 1, further comprising a measuring unit measuring a depth where the implant instrument inserts into vertebra.

6. The implant instrument of claim 1, wherein the first thread structure, the second thread structure, the outer thread or a combination thereof is a single thread or a multiple thread.

7. The implant instrument of claim 1, wherein the sleeve has an accommodating structure disposed on an outer surface thereof and defining a channel configured to accommodate the endoscope.

8. The implant instrument of claim 1, wherein the sleeve has a channel therein and configured to accommodate the endoscope, and the end of the endoscope is exposed out of an end of the channel close to the jaw part.

9. The implant instrument of claim 1, wherein the sleeve has a liquid inlet port disposed thereon and in communication with internal space thereof.

10. An implant instrument operative to grip and implant an intervertebral cage into a location between two adjacent spine bones, the intervertebral cage comprising a gripping end and a guiding end, and the implant instrument comprising:
a body comprising a grasp rod and a fixed rod, the fixed rod having a first end and a second end, the first end configured to assemble with the grasp rod, the fixed rod having a first guiding slot and a second guiding slot in communication with the second end, and the fixed rod having an outer thread near a side of the first end;
a gripper subassembly comprising a first connecting member, a first gripping member, a second connecting member and a second gripping member, the first connecting member connected to the first gripping member and disposed in the first guiding slot, the second connecting member connected to the second gripping member and disposed in the second guiding slot, the first connecting member having a first thread structure and the second connecting member having a second thread structure, the first gripping member having a first fastening slot and the second gripping member having a second fastening slot, wherein the first fastening slot and the second fastening slot are configured to form a jaw portion to grip the gripping end of the intervertebral cage;
a first control subassembly comprising a first rotating shaft and a second rotating shaft, the first rotating shaft connected to the second rotating shaft and sleeved outside the fixed rod, the first rotating shaft having an internal thread configured to screw with the first thread structure and the second rotating shaft having an internal thread configured to screw with the second thread structure; and
a second control subassembly comprising a sleeve and a gripping rotating shaft, the sleeve connected to the gripping rotating shaft and sheathed outside the fixed rod, the gripping rotating shaft having an internal thread configured to screw with the outer thread of the fixed rod, and the sleeve having a liquid inlet port disposed thereon and in communication with internal space thereof;
wherein while the first control subassembly is rotated, the first connecting member and the second connecting member respectively are moved to opposite directions along the first guiding slot and the second guiding slot to change a relative position between the jaw portion and the intervertebral cage so as to change an angle with respect to the location of the intervertebral cage.

11. The implant instrument of claim 10, further comprising an endoscope combined with the sleeve, and having an end provided with a camera and facing the jaw part.

12. The implant instrument of claim 11, wherein the sleeve has an accommodating structure disposed on an outer surface thereof and defining a channel configured to accommodate the endoscope.

13. The implant instrument of claim 11, wherein the sleeve has a channel therein and configured to accommodate the endoscope, and the end of the endoscope is exposed out of an end of the channel close to the jaw part.
